(19) United States
(12) Patent Application Publication
(10) Pub. No.: US 2011/0137199 A1
(43) Pub. Date: Jun. 9, 2011

(54) VISCERAL FAT MEASURING DEVICE

(75) Inventors: Hiromichi KARO, Kyoto-shi (JP); Takehiro HAMAGUCHI, Kyoto-shi (JP); Tomoya IJIRI, Kameoka-shi (JP); Yasuaki MURAKAWA, Kyoto-shi (JP); Shojiro OKU, Kyoto-shi (JP)

(73) Assignee: OMRON HEALTHCARE CO., LTD., Kyoto-shi (JP)

(21) Appl. No.: 13/024,955

(22) Filed: Feb. 10, 2011

Related U.S. Application Data
(63) Continuation of application No. PCT/JP2009/066407, filed on Sep. 18, 2009.

(30) Foreign Application Priority Data
Sep. 22, 2008 (JP) 2008-243168

(51) Int. Cl. A61B 5/053

(52) U.S. Cl. 600/547

(57) ABSTRACT

A visceral fat measuring device capable of simply and safely measuring a visceral fat amount is provided. In the visceral fat measuring device for calculating the visceral fat amount based on trunk measurement information, impedance information of the entire trunk, and impedance information of a surface layer of the trunk obtained by measuring a potential difference in the body axis direction of the trunk on the dorsal side of the trunk, a belt has a hollow pressed member pressed onto the dorsal side of the trunk, the pressed member having a pressed surface provided with electrodes, a wiring member including a circuit substrate connected to the electrodes for measuring the potential difference is accommodated inside the pressed member, the pressed member has flexibility in the vertical direction to the body axis direction so as to be curved along a surface shape of the dorsal side of the trunk, and when the pressed member is curved, an opposite surface to the pressed surface is extended relatively to the pressed surface and deflected.

### TECHNICAL FIELD

The present invention relates to a visceral fat measuring device.

### BACKGROUND ART

Conventionally, there is a known method of measuring a visceral fat amount from a tomographic image taken with using X ray CT and MRI. According to such a measuring method, although the visceral fat amount can be measured with high precision, large-sized facilities are required. Thus, measurement is only performed in medical treatment facilities where the X ray CT and the MRI are installed. Therefore, daily measurement of the visceral fat amount by such a measuring method is not realistic. Particularly, the X ray CT is capable of taking a finer image than the MRI. However, there is a known risk of radiation exposure.

Realization of a device capable of simply and noninvasively measuring the visceral fat amount is desired.

It should be noted that a related technology is disclosed in patent document 1.

### Related Art Document

### Patent Document

[Patent Document 1]: Japanese Unexamined Patent Publication No. 2002-369806

### DISCLOSURE OF THE INVENTION

### Problems to be Solved by the Invention

An object of the present invention is to provide a visceral fat measuring device capable of simply and noninvasively measuring a visceral fat amount.

### Means for Solving the Problem

In the present invention, the following means are adopted in order to solve the above problem.

That is, a visceral fat measuring device of the present invention is to calculate a visceral fat amount based on trunk measurement information serving as a basis for calculating a trunk sectional area in a section on an abdominal part of a trunk vertical to a body axis of the trunk, impedance information of the entire trunk obtained by applying an electric current from hands and legs to the trunk and measuring a potential difference in part of a surface of the trunk, and impedance information of a surface layer of the trunk obtained by winding a belt having a plurality of electrodes around the trunk so as to apply the electric current through the vicinity of the surface layer of the trunk and measure a potential difference in part of the surface of the trunk, wherein the belt has a hollow pressed member pressed onto the trunk, the pressed member having a pressed surface provided with a plurality of the electrodes, a wiring member including a circuit substrate connected to a plurality of the electrodes for measuring the potential difference is accommodated inside the pressed member, the pressed member has flexibility in the vertical direction to the body axis direction so as to be curved along a surface shape of the trunk, and when the pressed member is curved, an opposite surface to the pressed surface provided with a plurality of the electrodes is extended relatively to the pressed surface and deflected.

It should be noted that the “visceral fat amount” in the present invention includes indicators showing the visceral fat amount such as a visceral fat sectional area, a visceral fat volume, and a ratio of the visceral fat sectional area relative to an abdominal sectional area.

According to the present invention, the visceral fat amount can be measured from the trunk measurement information serving as the basis for calculating the trunk sectional area, the impedance information of the entire trunk, and the impedance information of the surface layer of the trunk. A circumferential length of a waist part (waist length) or vertical width and horizontal width of the trunk are taken as the trunk measurement information serving as the basis for calculating the trunk sectional area, and these can be easily measured. Since the impedance information can be obtained by measuring the potential difference in a state that an electric current is applied to a human body (a living body), the impedance information can be also easily obtained. Therefore, the visceral fat amount can be relatively easily and noninvasively measured.

According to the present invention, in order to measure the potential difference of the trunk and apply the electric current to the trunk through the vicinity of the surface layer of the trunk, a belt having electrodes is used. This belt has a hollow pressed member pressed onto the trunk, and a plurality of the electrodes is provided in a pressed surface of the pressed member. In a case where the belt formed in such a way is wound around a waist, the pressed member is desirably curved along a surface shape of the trunk in order to bring the electrodes into firm contact, and a circuit substrate for measuring the potential difference and the electrodes are desirably arranged at closer positions in order to reduce a measurement error in measuring the potential difference. In the present invention, when the pressed member is curved, an opposite surface to the pressed surface is extended relatively to the pressed surface and deflected. Thus, the pressed member can be curved without narrowing accommodation space inside. Therefore, a wiring member inside is suppressed from being abutted against an inner wall surface of the pressed member when the pressed member is curved. Thereby, the circuit substrate for measuring the potential difference and the electrodes can be arranged at closer positions, so that varied measurement results can be decreased, and measurement precision can be improved.

In a case where the potential difference in part of the surface of the trunk is measured, a potential difference on the dorsal side may be measured.

The opposite surface to the pressed surface may have a stretching portion having stretchability in the belt longitudinal direction and flexibility in the vertical direction to the body axis direction, and a non-stretching portion having neither stretchability nor flexibility, the wiring member may include a wiring portion having pliability, and a wiring portion having no pliability, the wiring portion having pliability may be arranged in inner space where the stretching portion is positioned, and the wiring portion having no pliability may be arranged in inner space where the non-stretching portion is positioned.

According to this configuration, when the pressed member is curved, a physical influence on the wiring member arranged inside can be further reduced.

In a case where the potential difference in part of the surface of the trunk is measured, a potential difference in the body axis direction of the trunk may be measured.

The pressed member may have gripping portions capable of gripping the pressed member at both ends in the belt longitudinal direction.

According to this configuration, by holding the gripping portions at both the ends of the pressed member and pressing the pressed member onto the dorsal, the pressed member can be pressed while being curved.

The gripping portions are preferably formed so as to support the pressed member in a state that fingers are extended along the belt longitudinal direction and palms are placed onto ends on the opposite surface to the pressed surface.

According to this configuration, the pressed member can be pressed onto the dorsal by the palms while being curved by finger tips at both the ends, so that a pressing task of the pressed member can be easily performed. Assuming measurement in an upright position, even in a case where there is only one measurer, a subject grips part of the belt, so that the measurement can be smoothly performed.

The gripping portions are preferably formed to be foldable relative to the pressed member along the belt longitudinal direction.

According to this configuration, when a user to which the belt is attached lies on a bed, the gripping portions can be suppressed from being nipped between the bed and a body so as to disturb the task and generate breakage. Even in highly narrow place such as a laboratory in a hospital, the device can be compactly stored.

The pressed member may be provided with locking means capable of locking a cable connecting the belt and a device main body so that the cable is extended substantially along any of the belt longitudinal direction.

By locking the cable in such a way, the cable can be prevented from disturbing the task.

A lean body sectional area excluding a fat may be calculated from the impedance information of the entire trunk, a subcutaneous fat sectional area may be calculated from the impedance information of the surface layer of the trunk, and a visceral fat sectional area may be calculated by subtracting the lean body sectional area and the subcutaneous fat sectional area from the trunk sectional area calculated from the trunk measurement information.

That is, the impedance of the entire trunk is largely influenced by an amount of lean body (viscera, muscles, and skeletons) excluding the fat. The lean body sectional area can be calculated from this impedance. The impedance of the surface layer of the trunk is largely influenced by an amount of a subcutaneous fat amount. The subcutaneous fat sectional area can be calculated from this impedance. It should be noted that a subcutaneous fat is generally accumulated in an area from sides to the dorsal side rather than the abdominal side of the trunk. Thus, by measuring the impedance on the dorsal side, the subcutaneous fat sectional area can be more precisely measured. With using the lean body sectional area and the subcutaneous fat sectional area obtained in such a way, by subtracting these areas from the trunk sectional area, the visceral fat sectional area can be obtained.

It should be noted that the above configurations can be combined and adopted as far as possible.

### Effect of the Invention

As described above, according to the present invention, the visceral fat amount can be simply and noninvasively measured.

### BRIEF DESCRIPTION OF THE DRAWINGS

FIG. 1 is a schematic view showing a state when impedance is measured.

FIG. 2 is a schematic view showing a state when impedance is measured.

FIG. 3 is an entire configuration diagram of a visceral fat measuring device according to an embodiment of the present invention.

FIG. 4 is a control block diagram of the visceral fat measuring device according to the embodiment of the present invention.

FIG. 5 is a perspective view of a pressed member of a belt in the visceral fat measuring device according to the embodiment of the present invention (Example 1).

FIG. 6 is a perspective view of the pressed member of the belt in the visceral fat measuring device according to the embodiment of the present invention (Example 1).

FIG. 7 is a perspective sectional view of the pressed member of the belt in the visceral fat measuring device according to the embodiment of the present invention (Example 1), in which part of the pressed member is removed.

FIG. 8 is a schematic view showing a state that the pressed member of the belt in the visceral fat measuring device according to the embodiment of the present invention (Example 1) is pressed.

FIG. 9 is a schematic view showing a state that the belt in the visceral fat measuring device according to the embodiment of the present invention (Example 1) is wound.

FIG. 10A is a schematic view for illustrating a configuration of a belt according to a conventional technology, showing a correct attachment state.

FIG. 10B is a schematic view for illustrating a configuration of the belt according to the conventional technology, showing an attachment state in a case where positions of electrodes are displaced.

FIG. 11 is a schematic view showing a state that a belt in the visceral fat measuring device according to the embodiment of the present invention (Example 2) is wound.

FIG. 12A is a plan view of a pressed member of a belt in the visceral fat measuring device according to the embodiment of the present invention (Example 3), showing a state that a cable is unlocked.

FIG. 12B is a plan view of the pressed member of the belt in the visceral fat measuring device according to the embodiment of the present invention (Example 3), showing a state that the cable is locked by one of locking means.

FIG. 12C is a plan view of the pressed member of the belt in the visceral fat measuring device according to the embodiment of the present invention (Example 3), showing a state that the cable is locked by the other locking means.

FIG. 13A is a schematic view for illustrating a configuration of the locking means, showing a state before locking.

FIG. 13B is a schematic view for illustrating the configuration of the locking means, showing a state after locking.

FIG. 14A is a schematic view for illustrating an example of how to pull out the cable.

FIG. 14B is a schematic view for illustrating an example of how to pull out the cable.

### BEST MODE FOR CARRYING OUT THE INVENTION

Modes for carrying out this invention will be described in detail as examples with reference to the drawings based on an embodiment. However, the scope of this invention is not limited to size, materials, shapes, relative arrangement, and the like of constituent elements described in this embodiment unless specifically described.

### Embodiment

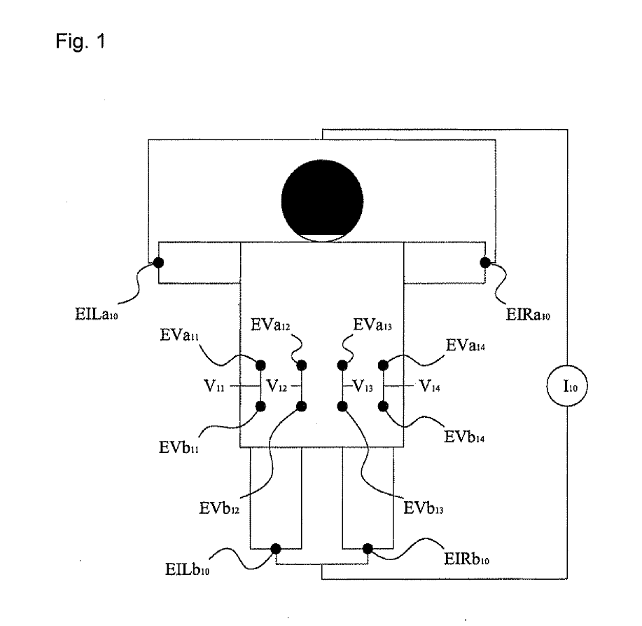

With reference to FIGS. 1 to 11, a visceral fat measuring device according to the embodiment of the present invention will be described.

(Measurement Principle of Visceral Fat)

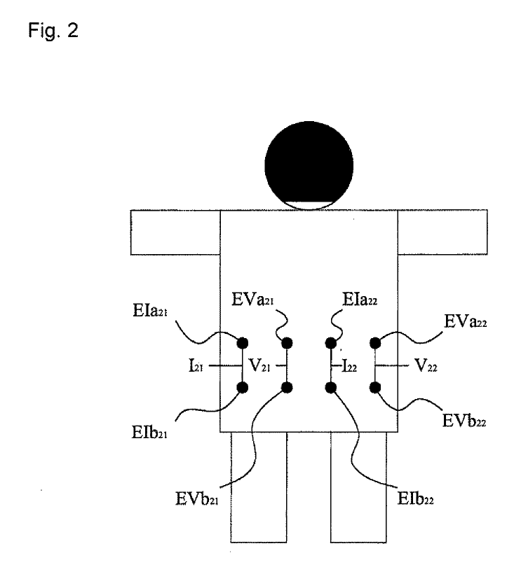

With reference to FIGS. 1 and 2, a measurement principle of a visceral fat in the visceral fat measuring device according to the embodiment of the present invention will be described. FIGS. 1 and 2 are schematic views showing states when impedance is measured. It should be noted that FIGS. 1 and 2 show states seen from the dorsal side of a user subjected to measurement of the visceral fat.

FIG. 1 shows the state in a case where impedance information of the entire trunk is obtained. As shown in the figure, electrodes EILa10, EIRa10 are respectively attached to both hands of the user subjected to the measurement of the visceral fat. Electrodes EILb10, EIRb10 are also respectively attached to both legs of the user. Pairs of electrodes provided side by side in the body axis direction of the trunk are attached at four points in the horizontal width direction of the trunk on the dorsal side of the trunk of the user. That is, the total of eight electrodes EVa11, EVb11, EVa12, EVb12, EVa13, EVb13, EVa14, EVb14 are attached.

In this state, an electric current I10 passing through the trunk is applied with using the electrodes EILa10, EIRa10, EILb10, EIRb10 respectively attached to the both hands and the both legs. A potential difference V11 is measured with using a pair of the electrodes EVa11, EVb11, a potential difference V12 is measured with using a pair of the electrodes EVa12, EVb12, a potential difference V13 is measured with using a pair of the electrodes EVa13, EVb13, and a potential difference V14 is measured with using a pair of the electrodes EVa14, EVb14. That is, the potential differences in part of a surface of the trunk are measured at the four points on the dorsal side.

Impedance Zt of the entire trunk is calculated from the potential differences measured in such a way. It should be noted that by measuring the potential differences V11, V12, V13, V14 at the four points and calculating the impedance of the entire trunk with using an average value thereof, an influence of varied fat distribution in the trunk, and the like can be reduced.

In a case where the electric current I10 is applied from the both hands and the both legs which are distant from the trunk, almost all the electric current I10 passes through a part where electric resistance is low, that is, a part other than a fat. Therefore, the impedance Zt of the entire trunk calculated from the potential differences V11, V12, V13, V14 measured with using such an electric current I10 is largely influenced by an amount of lean body (viscera, muscles, and skeletons) excluding the fat. Therefore, a lean body sectional area Sa (estimated value) can be calculated from this impedance Zt.

FIG. 2 shows the state in a case where impedance information of a surface layer of the trunk on the dorsal side of the trunk is obtained. As shown in the figure, pairs of electrodes provided side by side in the body axis direction of the trunk are attached at four points in the horizontal width direction of the trunk on the dorsal side of the trunk of the user. That is, the total of eight electrodes EIa21, EIb21, EVa21, EVb21, EIa22, EIb22, EVa22, EVb22 are attached.

In this state, an electric current I21 is applied with using a pair of the electrodes EIa21, EIb21, and an electric current I22 is applied with using a pair of the electrodes EIa22, EIb22. It should be noted that a current value of the electric current I21 and a current value of the electric current I22 are the same. A potential difference V21 is measured with using a pair of the electrodes EVa21, EVb21, and a potential difference V22 is measured with using a pair of the electrodes EVa22, Evb22. That is, the potential differences in part of the surface of the trunk are measured at the two points on the dorsal side.

Impedance Zs of the surface layer on the dorsal side of the trunk is calculated from the potential differences measured in such a way. It should be noted that by measuring the potential differences V21, V22 at the two points and calculating the impedance Zs of the surface layer of the trunk with using an average value thereof, an influence of varied subcutaneous fat and the like can be reduced. It should be noted that by switching a circuit so that the electrodes for applying the electric current serve as electrodes for measuring the potential differences, and the electrodes for measuring the potential differences serve as electrodes for applying the electric currents, the potential differences can be measured at the four points. In such a way, the influence of the varied subcutaneous fat and the like can be furthermore reduced.

In a case where the electric currents I21, I22 are applied by a pair of the electrodes attached at the positions on the back side of an abdominal part on the dorsal, almost all the electric currents I21, I22 pass through the surface layer of the trunk. Therefore, the impedance Zs of the surface layer of the trunk calculated from the potential differences V21, V22 measured with using such electric currents I21, I22 is largely influenced by an amount of a subcutaneous fat amount. Therefore, a subcutaneous fat sectional area Sb (estimated value) can be calculated from this impedance Zs.

Therefore, when a trunk sectional area (an area of a section on the abdominal part of the trunk vertical to a body axis of the trunk) is St, a visceral fat sectional area Sx is Sx=St−Sa−Sb. Thus, the visceral fat sectional area Sx can be calculated.

The trunk sectional area St can be calculated from a circumferential length of a waist part (waist length) or vertical width and horizontal width of the trunk (in the vicinity of the abdominal part). For example, in a case of calculating from the vertical width and the horizontal width of the trunk, when the horizontal width of the trunk is 2a, and the vertical width is 2b, the section of the trunk is substantially oval. Thus, the trunk sectional area is substantially “π×a×b”. However, this value is highly susceptible to an error. Thus, by multiplying a coefficient for correcting the error, a more precise trunk sectional area St can be obtained. With regard to this coefficient, for example based on a large number of X ray CT image samples, an optical value of α can be determined from a relationship between a trunk sectional area St′ obtained from the X ray CT images, and a value a, and a value b so as to satisfy “St′=α×π×a×b”.

Thereby, based on the horizontal width 2a and the vertical width 2b of the trunk, the trunk sectional area St(=α×π×a×b) with less error can be calculated. It should be noted that since the value α multiplied for correction may have an optimal value appropriately differentiated in accordance with gender, age, body height, weight, and the like (hereinafter, these are called as user information), by changing the value α in accordance with the user subjected to the measurement, the more precise trunk sectional area St can be calculated.

As described above, the lean body sectional area Sa can be calculated from the impedance Zt of the entire trunk. However, the lean body sectional area Sa cannot be calculated only with the impedance Zt of the entire trunk. That is, there is a need for converting a value proportional to size of the trunk obtained from the impedance Zt into the lean body sectional area Sa. More specifically, for example, the lean body sectional area Sa can be expressed as Sa=β×a×(1/Zt).

The value a is a half of the horizontal width of the trunk as described above, which is a value relating to the size of the trunk. This value is not limited to this. For example, (a×b) may be used so that values of the vertical width and the horizontal width of the trunk are reflected, the trunk sectional area St may be used, or the circumferential length of the waist part (the waist length) may be used.

The value β is a coefficient for converting into the lean body sectional area Sa, and an optimal value thereof can be determined from a large number of the X ray CT image samples as well as a case where the value α is determined. That is, based on a large number of the X ray CT image samples, the optimal value of β can be determined from a relationship between a lean body sectional area Sa′ obtained from the X ray CT images, and the value a, and the impedance Zt of the entire trunk of a person subjected to the X ray CT images so as to satisfy “Sa′=β×a(1/Zt)”.

Further, as described above, the subcutaneous fat sectional area Sb can be calculated from the impedance Zs of the surface layer of the trunk on the back side of the abdominal part on the dorsal. However, the subcutaneous fat sectional area Sb cannot be calculated only with the impedance Zs of the surface layer. That is, there is a need for converting a value proportional to the size of the trunk obtained from the impedance Zs into the subcutaneous fat sectional area Sb. More specifically, for example, the subcutaneous fat sectional area Sb can be expressed as Sb=γ×a×Zs.

The value a is the half of the horizontal width of the trunk as described above, which is the value relating to the size of the trunk. This value is not limited to this. For example, (a×b) may be used so that the values of the vertical width and the horizontal width of the trunk are reflected, the trunk sectional area St may be used, or the circumferential length of the waist part (the waist length) may be used.

The value γ is a coefficient for converting into the subcutaneous fat sectional area Sb, and an optimal value thereof can be determined from a large number of the X ray CT image samples as well as a case where the value α is determined. That is, based on a large number of the X ray CT image samples, the optimal value of γ can be determined from a relationship between a subcutaneous fat sectional area Sb′ obtained from the X ray CT images, and the value a, and the impedance Zs of the surface layer of the trunk of the person subjected to the X ray CT images so as to satisfy “Sb′=γ×a×Zs”.

It should be noted that the above values β and γ may have optical values appropriately differentiated in accordance with the user information as well as the value α used in a case where the sectional area of the abdominal part is determined. Therefore, by changing the values β and γ in accordance with the user subjected to the measurement, more precise lean body sectional area Sa and subcutaneous fat sectional area Sb can be calculated.

As described above, in the visceral fat measuring device according to the present embodiment, the visceral fat sectional area Sx is calculated from the trunk sectional area St, the lean body sectional area Sa calculated based on the impedance Zt of the entire trunk, and the subcutaneous fat sectional area Sb calculated based on the impedance Zs of the surface layer of the trunk.

That is, the visceral fat sectional area is expressed as Sx=St−Sa−Sb.

In this case, “St=α×π×a×b”, “Sa=β×a×(1/Zt)”, and “Sb=γ×a×Zs” are established. Then, the value a is the half of the horizontal width of the trunk, and the value b is a half of the vertical width of the trunk. The values α, β, γ are the coefficients obtained based on a large number of the X ray CT image samples for determining the optimal values of St, Sa, Sb. It should be noted that these coefficients can be changed in accordance with the user information as described above.

As clear from the above expression, the measured (calculated) visceral fat amount is the visceral fat sectional area. However, the visceral fat amount as a measurement result is not limited to the visceral fat sectional area but may be a ratio of the visceral fat sectional area relative to the trunk sectional area, or a visceral fat volume converted from the visceral fat sectional area.

It should be noted that as clear from the above expression, the measurement principle of the visceral fat in the visceral fat measuring device according to the embodiment of the present invention is based on a thought that the visceral fat sectional area Sx can be obtained by subtracting the lean body sectional area Sa and the subcutaneous fat sectional area Sb from the trunk sectional area St.

However, the visceral fat measuring device according to the present invention is not always limited to simple adoption of the above expression “Sx=St−Sa−Sb”, but also includes application of such a principle.

For example, the visceral fat sectional area Sx can be determined from “Sx=St−Sa−Sb+δ” (δ is a correction amount). That is, with similar methods to a case where the above values α, β, γ are determined, the correction amount δ can be added based on a large number of the X ray CT image samples.

The visceral fat sectional area Sx can be determined from Sx=St−F (Zt, Zs, a, b). It should be noted that F (Zt, Zs, a, b) is a function having Zt, Zs, a, b as parameters.

That is, a total value of the lean body sectional area Sa and the subcutaneous fat sectional area Sb has a correlation with the impedance Zt of the entire trunk, the impedance Zs of the surface layer of the trunk, and the size of the trunk (the vertical width and the horizontal width of the trunk in the present embodiment). Therefore, the total value of the lean body sectional area Sa and the subcutaneous fat sectional area Sb can be determined from the function F (Zt, Zs, a, b) having the values Zt, Zs, a, b as the parameters. It should be noted that this function F (Zt, Zs, a, b) can also be derived from a large number of the X ray CT image samples.

(Entire Configuration of Visceral Fat Measuring Device)

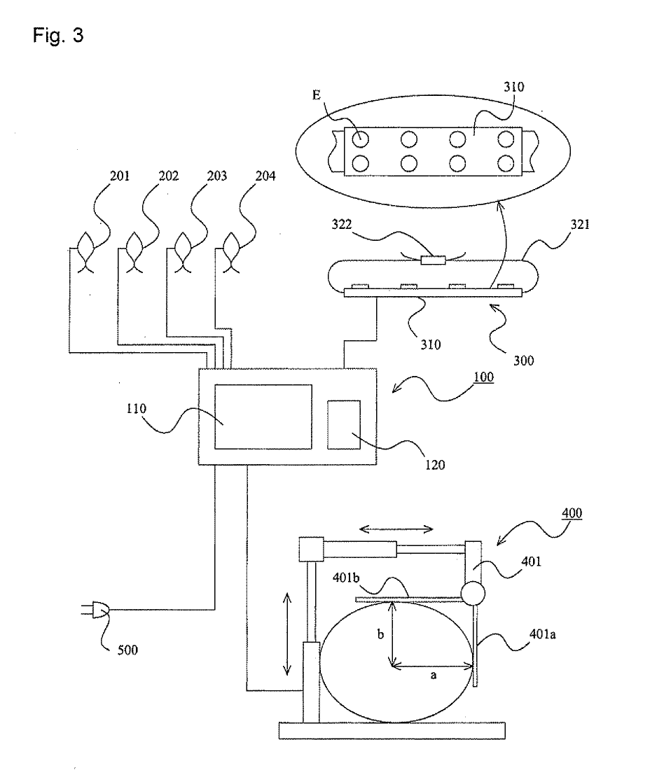

The entire configuration of the visceral fat measuring device according to the present embodiment will be described with reference to FIG. 3. FIG. 3 is an entire configuration diagram of the visceral fat measuring device according to the embodiment of the present invention.

The visceral fat measuring device according to the present embodiment is provided with a device main body 100, four clips 201, 202, 203, 204 for attaching electrodes to the hands and the legs, a belt 300 for attaching electrodes to the dorsal, a measuring unit 400 for measuring the vertical width and the horizontal width of the trunk, and a socket 500 for supplying electric power to the device main body 100.

The device main body 100 is provided with a display unit 110 for displaying various input information and the measurement result, and an operation unit 120 for turning on or off a power supply of the device main body 100 and inputting the various information.

The clips 201, 202, 203, 204 are respectively provided with the electrodes. By attaching these clips 201, 202, 203, 204 to the hands and the legs (preferably, wrists and ankles) so as to nip the hands and the legs, the electrodes can be closely attached to the hands and the legs. It should be noted that the electrodes respectively provided in the clips 201, 202, 203, 204 correspond to the electrodes EILa10, EIRa10, EILb10, EIRb10 shown in FIG. 1.

The belt 300 is provided with a pressed member 310 to be pressed onto the dorsal of the user subjected to the measurement, a belt portion 320 fixed to the both sides of the pressed member 310, and a buckle 330 for fixing the belt portion 320. The total of eight electrodes E are provided in the pressed member 310. By winding the belt 300 formed in such a way around a waist so that the pressed member 310 is abutted against a slightly upper part of coccyx, the eight electrodes E can be closely attached at positions on the back side of the abdominal part on the dorsal of the user. It should be noted that these eight electrodes E correspond to the eight electrodes EVa11, EVb11, EVa12, EVb12, EVa13, EVb13, EVa14, EVb14 shown in FIG. 1, and the eight electrodes EIa21, EIb21, EVa21, EVb21, EIa22, EIb22, EVa22, Evb22 shown in FIG. 2. That is, by switching the electric circuit in the device main body 100 between a case where the impedance Zt of the entire trunk is calculated and a case where the impedance Zs of the surface layer of the trunk is calculated, roles of the eight electrodes E can be changed.

The measuring unit 400 includes a cursor support portion 401 provided with a horizontal width measuring cursor portion 401a and a vertical width measuring cursor portion 401b. This cursor support portion 401 is formed to be movable in the up and down direction and the left and right direction. With using this measuring unit 400, for example, by moving the cursor support portion 401 to positions where the horizontal width measuring cursor portion 401a and the vertical width measuring cursor portion 401b are respectively brought into contact with sides and a navel and a periphery thereof in a state that the user lies on a bed, the horizontal width 2a and the vertical width 2b can be measured. It should be noted that in the present embodiment, the horizontal width 2a and the vertical width 2b of the trunk can be obtained as electric information (data) based on positional information of the cursor support portion 401 in the device main body 100. The trunk sectional area is calculated from the information relating to the horizontal width 2a and the vertical width 2b of the trunk obtained in such a way as described in the measurement principle of the visceral fat.

It should be noted that in the present embodiment, the visceral fat measuring device is provided with the measuring unit 400, and the vertical width and the horizontal width of the trunk and the trunk sectional area are automatically measured by this measuring unit 400. However, values obtained by other measurement devices or manual measurement and calculation can also be inputted into the device main body 100.

(Control Configuration of Visceral Fat Measuring Device)

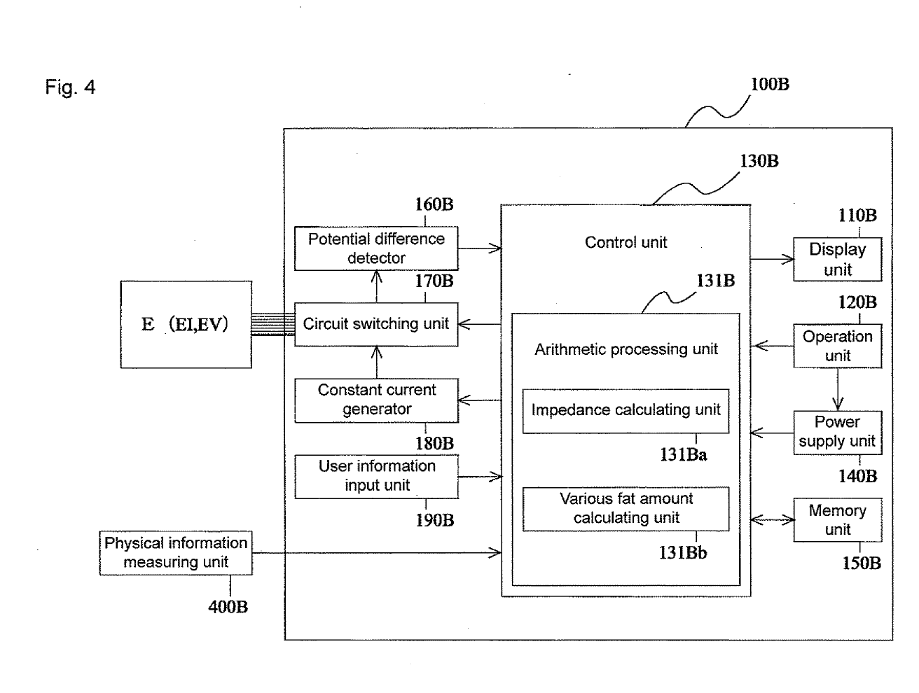

A control configuration of the visceral fat measuring device according to the present embodiment will be described with reference to FIG. 4. FIG. 4 is a control block diagram of the visceral fat measuring device according to the embodiment of the present invention.

In the visceral fat measuring device according to the present embodiment, a device main, body 100E is provided with a control unit (CPU) 130B, a display unit 110B, an operation unit 120B, a power supply unit 140B, a memory unit 150B, a potential difference detector 160B, a circuit switching unit 170B, a constant current generator 180B, and a user information input unit 190B.

The display unit 110B having a role of displaying input information from the operation unit 120B and the user information input unit 190B, the measurement result, and the like is formed by a liquid crystal display and the like. The operation unit 120B having a role of enabling the user or the like to input various information is formed by various buttons, a touchscreen, and the like. It should be noted that in the present embodiment, in addition to the input of the user information from the operation unit 120B, the user information is inputted from a barcode reader, a card reader, a USB memory, or the like via the user information input unit 190B.

The power supply unit 140B has a role of supplying the electric power to the control unit 130B and the like. When the power supply is turned on by the operation unit 120B, the electric power is supplied to the units, and when the power supply is turned off, the supply of the electric power is stopped. The memory unit 150B stores various data, programs, and the like for measuring the visceral fat.

The electrodes E respectively provided in the clips 201, 202, 203, 204 and the electrodes E provided in the belt are electrically connected to the circuit switching unit 170B provided in the device main body 100B. A physical information measuring unit 400B provided in the measuring unit 400 is electrically connected to the control unit 130B provided in the device main body 100B.

The control unit 130B has a role of controlling the entire visceral fat measuring device. The control unit 130B is provided with an arithmetic processing unit 131B. This arithmetic processing unit 131B is provided with an impedance calculating unit 131Ba for calculating impedance based on various information sent to the control unit 130B, and a various fat amount calculating unit 131Bb for calculating various fat amounts based on the calculated impedance.

The circuit switching unit 170B is for example formed by a plurality of relay circuits. This circuit switching unit 170B has a role of changing the electric circuit based on a command from the control unit 130B. That is, as described above, the circuit switching unit changes the electric circuit so as to have a circuit configuration shown in FIG. 1 in a case where the impedance information of the entire trunk is obtained, and to have a circuit configuration shown in FIG. 2 in a case where the impedance information of the surface layer of the trunk on the dorsal side is obtained.

The constant current generator 180B applies a high frequency current (of 50 kHz, 500 μA, for example) based on a command from the control unit 130B. More specifically, in a case of the electric circuit shown in FIG. 1, the electric current I10 is applied between the electrodes EILa10, EIRa10 and the electrodes EILb10, EIRb10. In a case of the electric circuit shown in FIG. 2, the electric currents I21, I22 are respectively applied between the electrode EIa21 and the electrode EIb21 and between the electrode EIa22 and the electrode EIb22.

The potential difference detector 160B detects a potential difference between predetermined electrodes while the electric current is applied by the constant current generator 180B. More specifically, in a case of the electric circuit shown in FIG. 1, the potential difference V11 is detected between the electrode EVa11 and the electrode EVb11, the potential difference V12 is detected between the electrode EVa12 and the electrode EVb12, the potential difference V13 is detected between the electrode EVa13 and the electrode EVb13, and the potential difference V14 is detected between the electrode EVa14 and the electrode EVb14, In a case of the electric circuit shown in FIG. 2, the potential difference V21 is detected between the electrode EVa21 and the electrode EVb21, and the potential difference V22 is detected between the electrode EVa22 and the electrode EVb22.

The potential difference information detected by the potential difference detector 160B is sent to the control unit 130B.

The physical information obtained by measurement by the measuring unit 400 is sent from the physical information measuring unit 400B to the control unit 130B of the device main body 100B. It should be noted that the physical information in the present embodiment is information relating to size of the horizontal width 2a and size of the vertical width 2b of the trunk.

In the arithmetic processing unit 131B in the control unit 130B, the impedance calculating unit 131Ba calculates the impedance Zt of the entire trunk and the impedance Zs of the surface layer of the trunk based on the potential difference information sent from the potential difference detector 160B. In the arithmetic processing unit 131B, the various fat amount calculating unit 131Bb calculates the various fat amounts (including the visceral fat sectional area) based on the calculated impedance Zt of the entire trunk and the impedance Zs of the surface layer of the trunk, the physical information sent from the physical information measuring unit 400B, and various information sent from the operation unit 120B and the user information input unit 190B.

Next, a measuring order in the visceral fat measuring device according to the present embodiment will be briefly described.

Firstly, the user subjected to the measurement of the visceral fat or a person who performs the measurement of the user turns on the power supply of the device main body 100 (100B) and inputs the user information. The vertical width and the horizontal width of the trunk of the user are measured by the measuring unit 400. Thereby, the information relating to the horizontal width 2a and the vertical width 2b of the trunk of the user is sent to the device main body 100 (100B). It should be noted that in the device main body 100 (100B), the trunk sectional area St (=α×π×a×b) is calculated based on the information. It should be noted that the value a is read from the memory unit 150B.

Next, the clips 201, 202, 203, 204 are attached to the hands and the legs of the user and the belt 300 is wound around the waist of the user. The measurement of the impedance is started.

In the present embodiment, firstly, the circuit switching unit 170B controls so as to have the electric circuit shown in FIG. 1. Thereby, the impedance Zt of the entire trunk is calculated by the impedance calculating unit 131Ba of the control unit 130B. The various fat amount calculating unit 131Bb calculates the lean body sectional area Sa (=β×a×(1/Zt)) from this calculated impedance Zt, the value a obtained by the measurement by the measuring unit 400, and the value β stored in the memory unit 150B.

Next, the circuit switching unit 170B controls so as to have the electric circuit shown in FIG. 2. Thereby, the impedance Zs of the surface layer of the trunk is calculated by the impedance calculating unit 131Ba of the control unit 130B. The various fat amount calculating unit 131Bb calculates the subcutaneous fat sectional area Sb (=γ×a×Zs) from this calculated impedance Zs, the value a obtained by the measurement by the measuring unit 400, and the value γ stored in the memory unit 150B.

The control unit 130B calculates the visceral fat sectional area Sx (=St−Sa−Sb) from the trunk sectional area St, the lean body sectional area Sa, and the subcutaneous fat sectional area Sb obtained as described above by the arithmetic processing unit 131B, and displays the values of the visceral fat sectional area Sx and the like on the display unit 110 (110B) as the measurement result. It should be noted that although a case where the various fat amount calculating unit determines the visceral fat sectional area Sx with using “Sx=St−Sa−Sb” is described in this measuring order, the visceral fat sectional area Sx may be determined with using “Sx=St−Sa−Sb+δ”, “Sx=St−F (Zt, Zs, a, b), or the like as described in the measurement principle of the visceral fat.

(Belt)

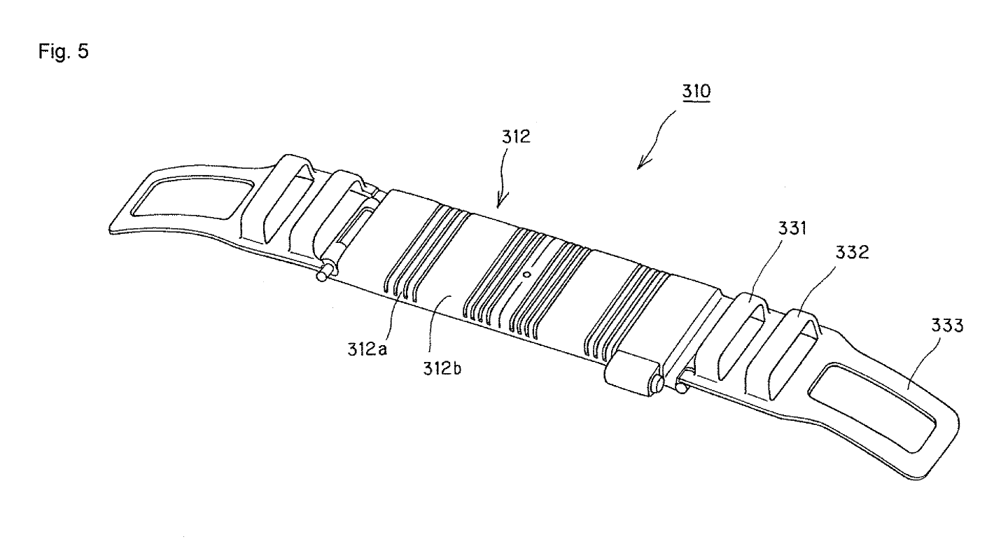

The belt will be described further in detail with reference to FIGS. 5 to 10B.

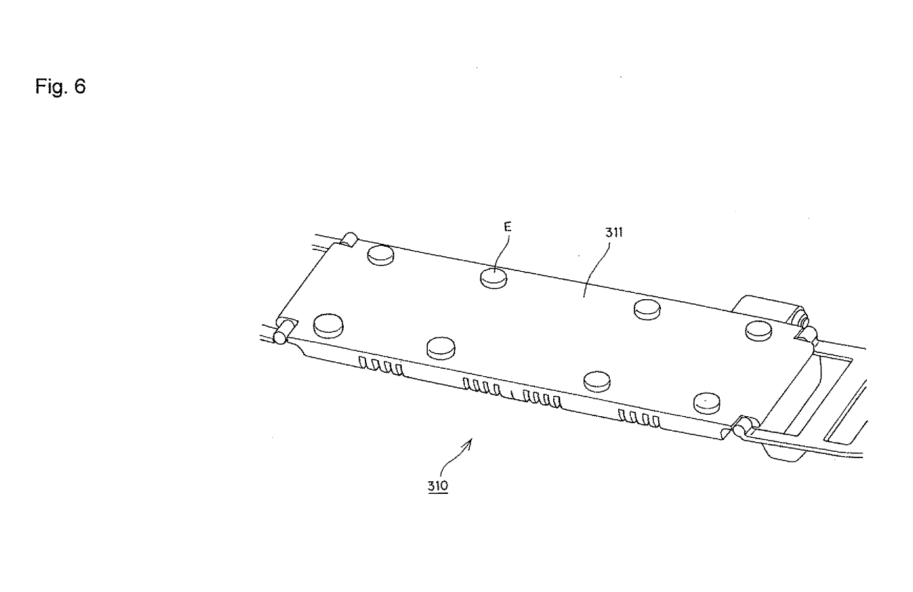

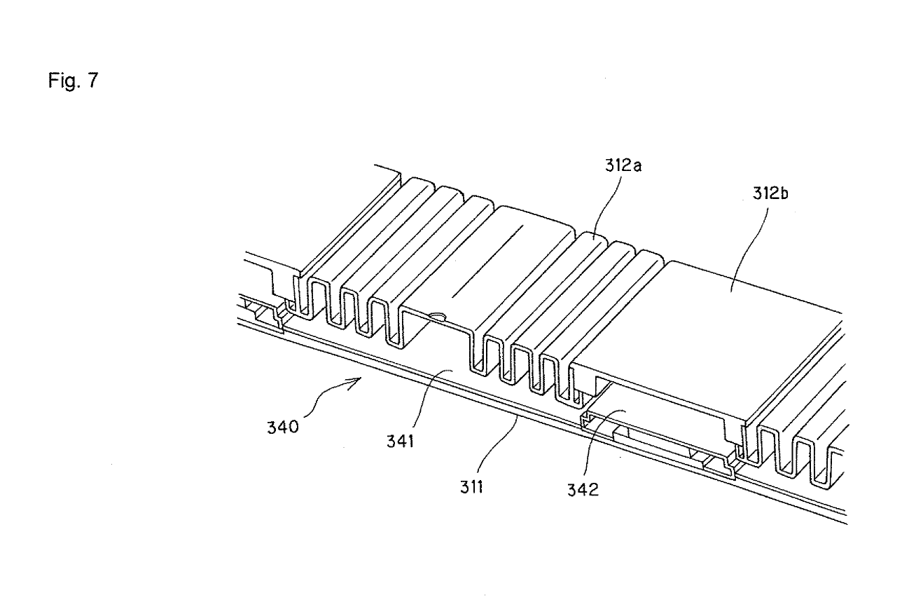

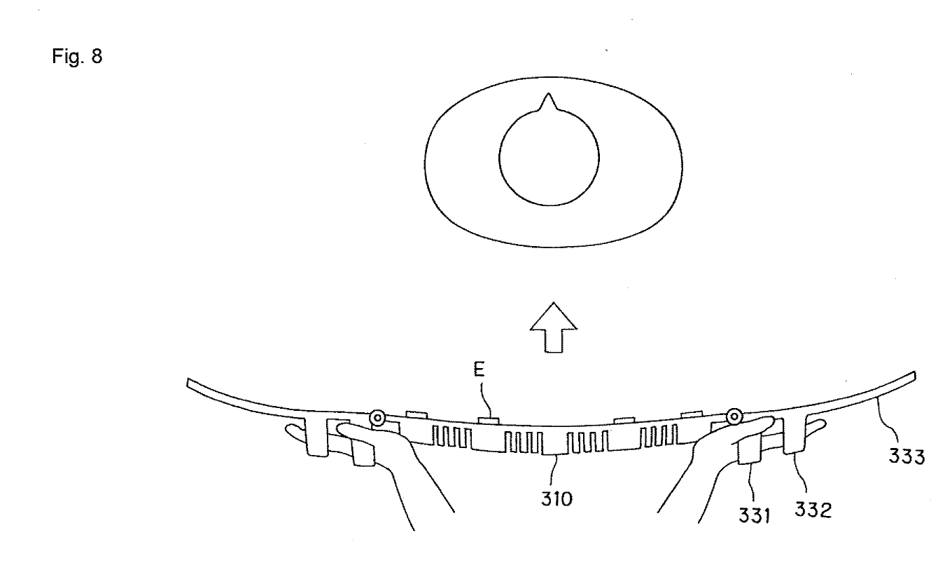

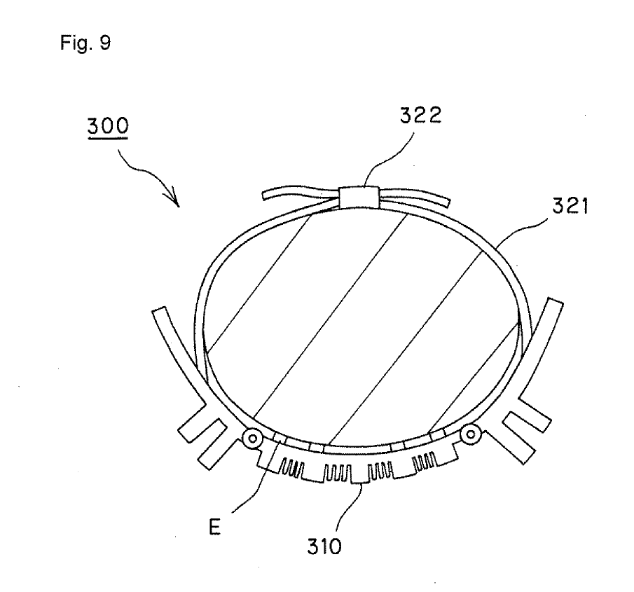

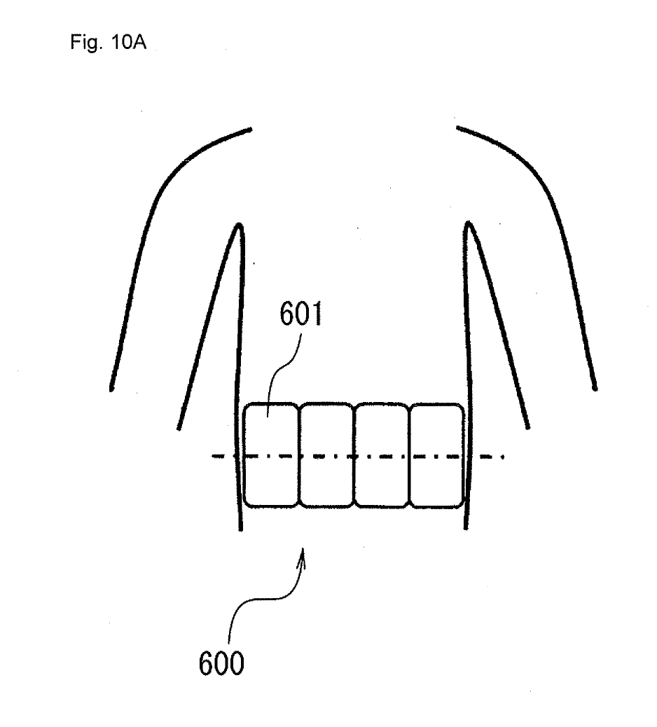

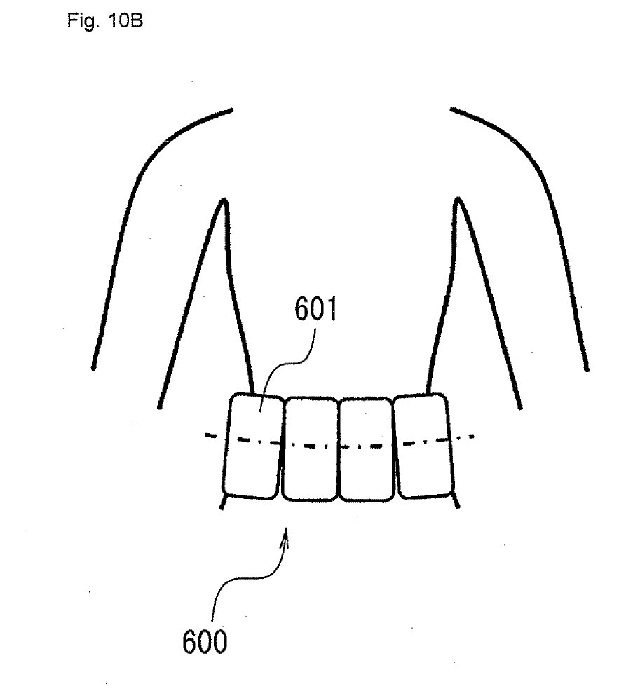

Example 1 of the belt will be described with reference to FIGS. 5 to 10B. FIG. 5 is a perspective view of the pressed member of the belt in the visceral fat measuring device according to the embodiment of the present invention (Example 1). FIG. 6 is a perspective view of the pressed member of the belt in the visceral fat measuring device according to the embodiment of the present invention (Example 1), which is FIG. 5 seen from the back side. FIG. 7 is a perspective sectional view of the pressed member of the belt in the visceral fat measuring device according to the embodiment of the present invention (Example 1), in which part of the pressed member is removed. FIG. 8 is a schematic view showing a state that the pressed member of the belt in the visceral fat measuring device according to the embodiment of the present invention (Example 1) is pressed. FIG. 9 is a schematic view showing a state that the belt in the visceral fat measuring device according to the embodiment of the present invention (Example 1) is wound. FIG. 10A is a schematic view for illustrating a configuration of a belt according to a conventional technology, showing a correct attachment state, and FIG. 10B is a schematic view for illustrating a configuration of the belt according to the conventional technology, showing an attachment state in a case where positions of electrodes are displaced.

The belt 300 according to the present embodiment is provided with the pressed member 310 to be pressed onto a position on the back side of the abdominal part on the dorsal of the user, belt portions 321 respectively fixed to the both sides of the pressed member 310, and a buckle 322 for fixing the belt portions 321.

The pressed member 310 is a flat band plate shape member extending in the longitudinal direction of the belt 300, and having a hollow part inside. The eight electrodes E are provided in a surface (a pressed surface) 311 of the pressed member pressed onto the dorsal of the user so as to form pairs in the short-side direction of the belt 300. The pressed surface 311 is made of a resin material or the like, and has flexibility in the directions other than the short-side direction (the body axis direction). Therefore, when the pressed member 310 is pressed onto the dorsal and curved, the pressed member is not deflected in the body axis direction. In an opposite surface 312 to the pressed surface 311, concave-convexo surface portions 312a (stretching portions) made of flexible members such as elastomers and flat surface portions (non-stretching portions) 312b made of rigid materials are formed alternately in the longitudinal direction.

In the concave-convexo surface portions 312a, concave parts and convex parts extending in the short-side direction are provided alternately and continuously in the longitudinal direction. The concave-convexo surface portion has a waving geometry in the longitudinal direction as a whole. With such a geometry, the concave-convexo surface portion 312a has stretchability in the longitudinal direction and flexibility in the vertical direction to the longitudinal direction. When the pressed member 310 is curved, the flat surface portion 312b is not stretched or deflected but the concave-convexo surface portion 312a is extended around the waist and deflected along a shape of a back surface of the trunk.

Gripping portions 331, 332, 333 for the user himself/herself or an assistant to grip the pressed member 310 when the belt 300 is attached to the waist are provided at both ends of the pressed member 310 in the longitudinal direction.

The gripping portions 331, 332 are mainly used by the assistant and formed into a handle shape. The gripping portions 331, 332 can be lifted up by gripping handle shape parts thereof, or lifted up by extending and inserting finger tips into holes of the handle shape parts. In a case where fingers are inserted into the holes of the handle shape parts, palms are placed onto both the ends of the pressed member 310. Therefore, as shown in FIG. 8, by inserting the hands into the gripping portions 331, 332 so as to grip the pressed member 310 and pressing both the ends onto the dorsal by the palms while curving the pressed member 310 by the finger tips, the belt can be easily attached.

Larger holes are provided in the gripping portions 333 so that the gripping portions can be firmly caught by the hands, and the gripping portions are mainly used when the user himself/herself attaches the belt 300 to the waist.

Various wiring members 340 such as circuit substrates and a cable for measuring the impedance are accommodated inside the hollow part of the pressed member 310. The accommodated wiring members include wiring members 341 having pliability such as flexible printed circuits (FPC) and flexible fiat cables (FFC), and wiring member 342 having no pliability such as rigid substrates. The wiring members 341 having pliability are arranged on the inner side of the concave-convexo surface portions 312a to be deformed when the pressed member 310 is curved, and the wiring members 342 having no pliability are arranged on the inner side of the flat surface portions 312b to be not deformed when the pressed member 310 is curved. Thereby, when the pressed member 310 is curved, the various wiring members 340 are not physically influenced.

For example in a technology described in patent document 1, circuits for measuring the potential difference are provided outside of an electrode belt. However, with a method of calculating a body fat by measuring the impedance, it can be said that the circuit substrates are preferably arranged in the vicinity of the electrodes, that is, the circuit substrates are preferably built into the electrode belt in order to suppress variation in the measurement in the electric circuits. However, considering that space is formed inside the electrode belt and the circuit substrates in which electronic parts are installed are built into the electrode belt, when the electrode belt is wound around the user, distortion is generated between an inner circumferential surface of the electrode belt (a surface in contact with a human body) and an outer circumferential surface (an outer appearance surface) by a circumferential difference due to a R shape of the trunk. Thus, it can be thought that problems that the space inside the electrode belt is crushed, and elasticity of the electrode belt is lowered so as to decrease a handling property are generated.

Meanwhile, with the belt in the visceral fat measuring device according to the present embodiment, as described above, the outer surface (the opposite surface to the pressed surface) is extended when the pressed member is curved, so that the pressed member can be curved without narrowing inner space where the circuit parts and the like are accommodated. Thus, there is no fear that the circuit parts and the like inside are abutted against an inner wall surface of the pressed member when the pressed member is curved. Therefore, a configuration that the circuit parts and the like are built inside the belt can be adopted, and the circuit substrates for measuring the potential difference and the electrodes are arranged at closer positions, so that measurement precision can be improved.

There is a known impedance meter 500 mounted on an upper surface of the abdominal part of the trunk of the user for measuring the impedance as shown in FIGS. 10A and 10B. However, this impedance meter 600 is formed by coupling blocks 601 provided with the electrodes by flexible members. For example, as shown in FIG. 10B, in a case where the waist of the user is highly constricted, the entire device is also deflected in the body axis direction, and contact positions between the electrodes and the human body and contact states are sometimes not uniform.

Meanwhile, with the belt in the visceral fat measuring device according to the present embodiment, as described above, the pressed member has no flexibility in the body axis direction. Thus, as described above, even in a case where the waist part is highly constricted, deformation in which the contact positions of the electrodes are displaced from a position in the section serving as a measurement reference (a navel position) is suppressed from being generated.

Next, Example 2 of the belt will be described with reference to FIG. 11. FIG. 11 is a schematic view showing a state that a belt in the visceral fat measuring device according to the embodiment of the present invention (Example 2) is wound.

In a belt 300a according to Example 2, gripping portions 331a are formed to be foldable relative to a pressed member 310a along the longitudinal direction (the direction around the waist) of the belt 300a. It should be noted that although movable parts of the gripping portions 331a are pivotable on supports in this example, the present invention is not limited to this. Not only the gripping portions 331a but also gripping portions 332 may be formed to be foldable.

According to such a configuration, when the user to which the belt 300a is attached lies on a bed 7, the gripping portions 331a can be suppressed from being nipped between the bed 7 and a body so as to disturb a task and generate breakage.

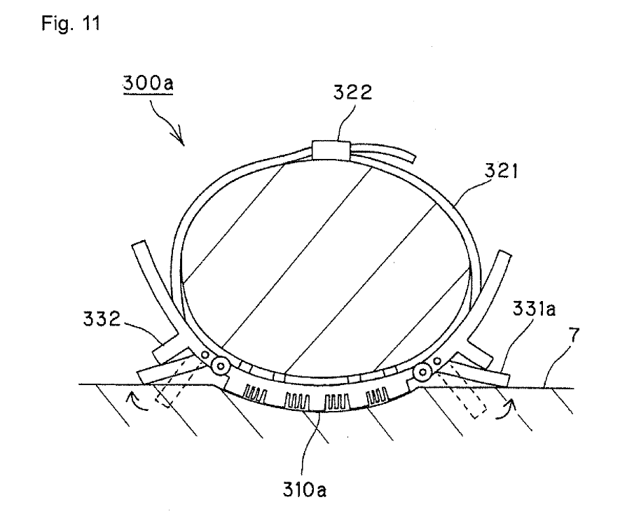

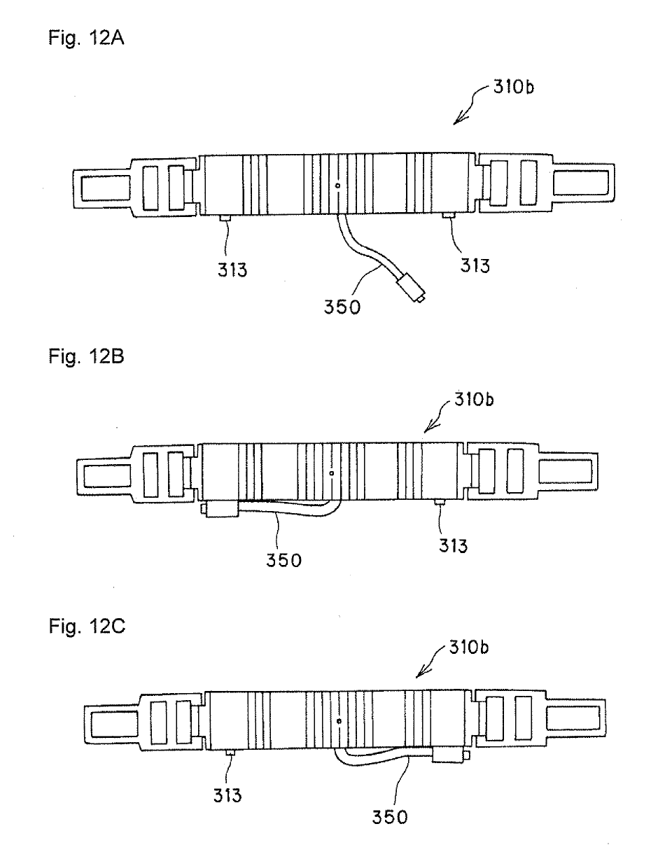

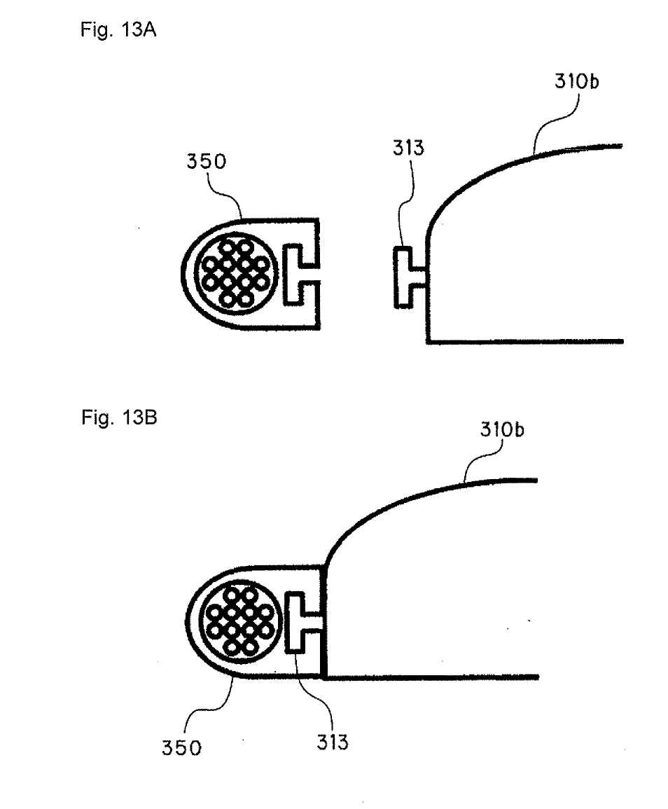

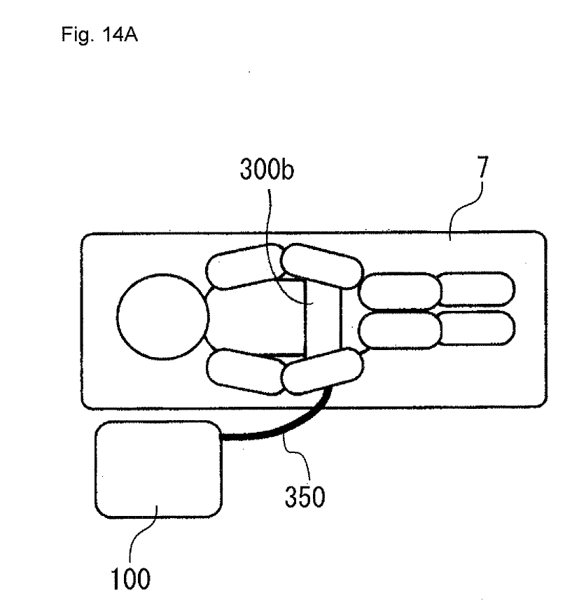

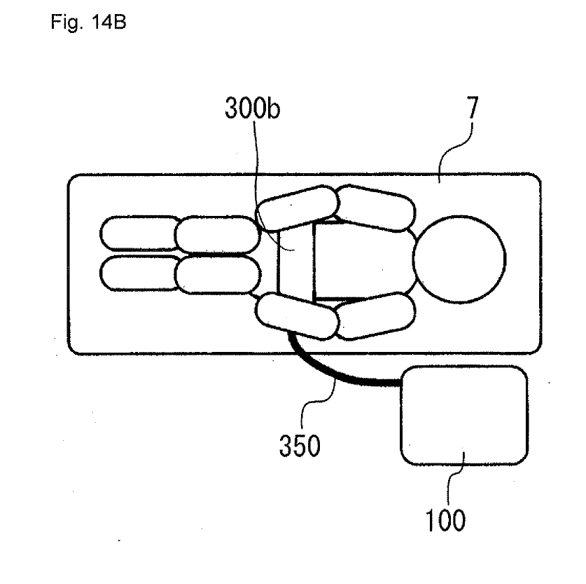

Next, Example 3 of the belt will be described with reference to FIGS. 12A to 14B. FIG. 12A is a plan view of a pressed member of a belt in the visceral fat measuring device according to the embodiment of the present invention (Example 3), showing a state that a cable is unlocked, FIG. 12B is a plan view of the pressed member of the belt in the visceral fat measuring device according to the embodiment of the present invention (Example 3), showing a state that the cable is locked by one of locking means, and FIG. 12C is a plan view of the pressed member of the belt in the visceral fat measuring device according to the embodiment of the present invention (Example 3), showing a state that the cable is locked by the other locking means. FIG. 13A is a schematic view for illustrating a configuration of the locking means, showing a state before locking, and FIG. 13B is a schematic view for illustrating the configuration of the locking means, showing a state after locking. FIGS. 14A and 14B are schematic views for illustrating examples of how to pull out the cable, respectively showing states that the device main body is differently arranged.

A belt 300b according to Example 3 is provided with the locking means capable of locking a cable 350 connecting the various wiring members accommodated in a pressed member 310b and the device main body 100 so that the cable is extended substantially along any of the belt longitudinal direction. In this example, sliding type locking mechanisms as shown in FIGS. 13A and 13B are provided as the locking means. That is, rail shape convex portions 313 extending in the longitudinal direction are provided in the pressed member 310b, and groove portions corresponding to shapes of the rail shape convex portions 313 are provided in the cable 350. By fitting the rail shape convex portions 313 into the groove portions, the cable 350 is locked onto the pressed member 310b. Such locking mechanisms are respectively provided at both ends of the pressed member 310b.

As shown in FIGS. 14A and 14B, a relationship between the direction in which the user lies and arrangement of the device main body 100 is sometimes differentiated due to situations of hospitals or the like. The unlocked and extended cable 350 is sometimes disturbing the task. Therefore, by locking the cable in accordance with the relationship between the direction in which the user lies and the arrangement of the device main body 100 relative to the arranged cable in such a way, the cable is prevented from disturbing the task, so that workability can be improved.

### DESCRIPTION OF SYMBOLS

100, 100B: Device main body

110, 110B: Display unit

120, 120B: Operation unit

130B: Control unit

131B: Arithmetic processing unit

131Ba: Impedance calculating unit

131Bb: Various fat amount calculating unit

140B: Power supply unit

150B: Memory unit

160B: Potential difference detector

170B: Circuit switching unit

180B: Constant current generator

190B: User information input unit

201, 202, 203, 204: Clip

300: Belt

310: Pressed member

311: Pressed surface

312: Opposite surface to pressed surface

312a: Concave-convexo surface portion

312b: Flat surface portion

321: Belt portion

322: Buckle

331, 332, 333: Gripping portion

340: Wiring member

400: Measuring unit

400B: Physical information measuring unit

401: Cursor support portion

401a: Horizontal width measuring cursor portion

401b: Vertical width measuring cursor portion

500: Socket

E: Electrode

1-9. (canceled)

10. A visceral fat measuring device for calculating a visceral fat amount based on trunk measurement information serving as a basis for calculating a trunk sectional area in a section on an abdominal part of a trunk vertical to a body axis of the trunk, impedance information of the entire trunk obtained by applying an electric current from hands and legs to the trunk and measuring a potential difference in part of a surface of the trunk, and impedance information of a surface layer of the trunk obtained by winding a belt having a plurality of electrodes around the trunk so as to apply the electric current through the vicinity of the surface layer of the trunk and measure a potential difference in part of the surface of the trunk, wherein the belt has a hollow pressed member pressed onto the trunk, the pressed member having a pressed surface provided with a plurality of the electrodes, a wiring member including a circuit substrate connected to a plurality of the electrodes for measuring the potential difference is accommodated inside the pressed member, due to an opposite surface to the pressed surface provided with a plurality of the electrodes having a stretching portion having stretchability in the belt longitudinal direction and flexibility in the vertical direction to the body axis direction, and a non-stretching portion having neither stretchability nor flexibility, the pressed member has flexibility in the vertical direction to the body axis direction so as to be curved along a surface shape of the trunk, when the pressed member is curved, the opposite surface to the pressed surface provided with a plurality of the electrodes is extended relatively to the pressed surface and deflected so as not to narrow inner space where the wiring member is accommodated, a wiring portion having pliability of the wiring member is arranged in the inner space where the stretching portion is positioned, and a wiring portion having no pliability of the wiring member is arranged in the inner space where the non-stretching portion is positioned.

11. The visceral fat measuring device according to claim 10, wherein the stretching portion is formed by a concave-convexo surface portion in which a concave part and a convex part extending in the short-side direction are provided alternately and continuously in the longitudinal direction, the non-stretching portion is formed by a flat surface portion, and due to extension of the opposite surface to the pressed surface by extension of the concave-convexo surface portion when the pressed member is curved, the pressed member is curved without narrowing the inner space where the wiring member is accommodated.
